(19) United States
(12) Patent Application Publication
(10) Pub. No.: US 2011/0064711 A1
(43) Pub. Date: Mar. 17, 2011

(54) COMPOSITIONS CONTAINING COENZYME Q-10 AND AN ANTIOXIDANT

(76) Inventor: Thomas Eidenberger, Steyr (AT)

(21) Appl. No.: 12/879,756

(22) Filed: Sep. 10, 2010

Related U.S. Application Data
(60) Provisional application No. 61/241,603, filed on Sep. 11, 2009.

(51) Int. Cl. A61K 38/43; A61K 31/122; A61P 25/16; A61P 25/06

(52) U.S. Cl. 424/94.1; 514/678

(57) ABSTRACT

The invention describes compositions of a reduced form of coenzyme Q-10 (CoQ10) or CoQ10 or both and an antioxidant that is an anthocyanin extract, a melanin or a compound having at least one thiol (—SH) group.

### CROSS REFERENCE TO RELATED APPLICATIONS

This application claims priority to U.S. Provisional Patent Application Ser. No. 61/241,603, filed Sep. 11, 2009 entitled “Compositions Containing Coenzyme Q-10 and an Antioxidant” (attorney docket number OMCA-038/PROV), U.S. patent application Ser. No. 12/510,395, entitled “Sesame Derived Pigments”, filed Jul. 28, 2009 (attorney docket number OMCA-028/US), which claims benefit under benefit under 35 U.S.C. §119(e) to U.S. Provisional Patent Application No. 61/083,994, entitled “Sesame Seed Derived Pigments”, filed Jul. 28, 2008 (attorney docket number OMCA-028/PROV), the contents of which are both incorporated herein in their entirety for all purposes.

### FIELD OF THE INVENTION

The invention relates generally to unique combinations of a reduced form of coenzyme Q-10 (CoQ10) and an antioxidant that is an anthocyanin extract, a melanin or a compound having at least one thiol (—SH) group.

### BACKGROUND OF THE INVENTION

CoQ-10 (coenzyme Q10) is a fat-soluble quinone, a benzoquinone that is structurally similar to vitamin K and commonly known as ubiquinone. CoQ-10 is found in most living organisms, and is essential for the production of cellular energy. CoQ-10 (2,3 dimethyl-5 methyl-6-decaprenyl benzoquinone) is an endogenous antioxidant found in small amounts in meats and seafood. Although CoQ-10 is found in all human cells, the highest concentrations of CoQ-10 occur in the heart, liver, kidneys, and pancreas. It is found naturally in the organs of many mammalian species.

CoQ-10 is an important nutrient because it lies within the membrane of a cell organelle called the mitochondria. Mitochondria are known as the “power house” of the cell because of their ability to produce cellular energy, or ATP, by shuttling protons derived from nutrient breakdown through the process of aerobic (oxygen) metabolism. CoQ-10 also has a secondary role as an antioxidant. CoQ-10, due to the involvement in ATP synthesis, affects the function of almost all cells in the body, making it essential for the health of all human tissues and organs. CoQ-10 particularly effects the cells that are the most metabolically active: heart, immune system, gingiva, and gastric mucosa

CoQ-10 is sparingly soluble in most hydrophilic solvents such as water. Therefore, CoQ-10 is often administered in a powdered form, as in a tablet or as a suspension. However, delivery of CoQ-10 by these methods limits the bioavailability of the material to the individual.

Several clinical trials have shown CoQ-10 to be effective in supporting blood pressure and cholesterol levels. Furthermore, CoQ-10 has also been shown to improve cardiovascular health. CoQ-10 has been implicated as being an essential component in thwarting various diseases such as certain types of cancers. These facts lead many to believe that CoQ-10 supplementation is vital to an individual's well being.

Reduced benzoquinones are known to be effective reductants for oxygen or lipid radicals. Some studies have shown that reduced CoQ-10 (ubiquinol) is an effective antioxidant. In fact, reduced CoQ-10 now appears to function as part of a complex chain of antioxidant activity. Apparently, reduced CoQ-10 plays a role in the reduction of radicals of alpha-tocopherol and ascorbate formed when these antioxidants are oxidized by oxygen or carboxyl radicals present in physiological systems. There are no known enzymes for direct reduction of a tocopheryl radical or an external ascorbate radical, but there are enzymes in all membranes that can reduce CoQ-10 and thus reduced CoQ-10 can subsequently reduce the tocopheryl or ascorbate radicals to provide tocopherol or ascorbate. Without the support of enzymes to reduce CoQ-10, the reduced coQ-10 would not be a very effective antioxidant as the semiquinone formed by interaction with lipid or oxygen radicals is readily autooxidized with formation of a superoxide radical.

Therefore, a need exists for methods and compositions that provide reduced CoQ-10 in a form that can be assimilated and retains antioxidant activity.

### BRIEF SUMMARY OF THE INVENTION

The present invention surprisingly provides compositions of coenzyme Q-10 (CoQ-10) and/or reduced CoQ-10 with an antioxidant that is an anthocyanin extract, a melanin, a compound having at least one thiol (—SH) group or mixtures thereof.

The compositions of the invention can further include various carriers and additives, such as additional suitable antioxidants and/or vitamins.

The present invention further provides methods to treat various conditions associated with decreased levels of CoQ-10, such as mitochondrial related diseases and disorders, Parkinson's disease, Prater-Willey syndrome, migraine headaches or headaches by administering to the individual in need thereof, an effective amount of any of the compositions disclosed herein.

In still another aspect, the present invention also provides packaged neutraceuticals that are disclosed herein.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 depicts the reduced CoQ-10 of Example 1.

FIG. 2 depicts the ratio of reduced CoQ-10 to CoQ-10 in oxidized form of Example 2.

### DETAILED DESCRIPTION

In the specification and in the claims, the terms “including” and “comprising” are open-ended terms and should be interpreted to mean “including, but not limited to. . . . ” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

As used herein, “subject” means mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

As used herein, “administering” or “administration” includes any means for introducing a composition of the invention into the body, preferably into the systemic circulation. Examples include but are not limited to oral, buccal, sublingual, pulmonary, transdermal, transmucosal, as well as subcutaneous, intraperitoneal, intravenous, and intramuscular injection.

The term “carrier” is intended to include any material, liquid or solid, that can be utilized to administer the compositions of the invention. This can include emulsions, suspensions, liquids, and food products and described herein.

The compositions of the invention can be administered in a therapeutically effective amount via a pharmaceutically acceptable method.

The term “therapeutically effective amount” is intended to mean an amount of a compound that, when administered to a subject for treating a disease, is sufficient to effect such treatment for the disease. The “therapeutically effective amount” will vary depending on the compound, the disease state being treated, the severity or the disease treated, the age and relative health of the subject, the route and form of administration, the judgment of the attending medical or veterinary practitioner, and other factors.

For purposes of the present invention, “treating” or “treatment” describes the management and care of a patient for the purpose of combating the disease, condition, or disorder. The terms embrace both preventative, i.e., prophylactic, and palliative treatments. Treating includes the administration of a compound of present invention to prevent the onset of the symptoms or complications, alleviating the symptoms or complications, or eliminating the disease, condition, or disorder.

A compound is administered to a patient in a therapeutically effective amount. A compound can be administered alone or as part of a pharmaceutically acceptable composition. In addition, a compound or composition can be administered all at once, as for example, by a bolus injection, multiple times, such as by a series of tablets, or delivered substantially uniformly over a period of time, as for example, using transdermal delivery. Further, the dose of the compound can be varied over time. A compound can be administered using an immediate release formulation, a controlled release formulation, or combinations thereof. The term “controlled release” includes sustained release, delayed release, and combinations thereof.

The term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. The preparation can be enclosed in ampoules, disposable syringes or multiple dose vials made of glass or plastic.

These compositions can take the form of solutions, suspensions, emulsion, tablets, pills, capsules, powders, sustained-release formulations and the like. The composition can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

The amount of the composition of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test bioassays or systems.

Administration of the doses recited above can be repeated. In one embodiment, the doses recited above are administered 1 to 7 times per week. The duration of treatment depends upon the patient's clinical progress and responsiveness to therapy.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

“Food” or “food product” refers to any solid or liquid edible substance that can be consumed by a subject. Such food products include baked goods, snack foods (candy, gum, candy bars, etc.), dairy products (ice cream, yogurt) and beverages (soda, milk, coffee, tea, etc.) and beverage mixes.

The present invention surprisingly provides solutions of coenzyme Q-10 (CoQ-10) and/or reduced CoQ-10 with an antioxidant that is an anthocyanin, a melanin, a compound having at least one thiol (—SH) group or mixtures thereof. The compositions of the invention can be delivered in any manner as described above.

In one embodiment the antioxidant is an anthocyanin, such as an anthocyanin extract, as a stabilizing agent for the CoQ-10 or reduced CoQ-10.

In one aspect, the present invention provides a stabilized anthocyanin extract composition that includes an anthocyanin extract and a stabilizing compound having at least one —SH group. Suitable examples of stabilizing compounds include (reduced) glutathione, dihydrolipoic acid, cysteine, yeast extract and mixtures thereof.

The term “anthocyanin” as used herein is intended to include both glycosylated anthocyanins (anthocyanosides) as well as the aglycon of the anthocyanoside (anthocyanidin). Throughout the specification, reference to the aglyconic anthocyanidin will often be made but should in no way be construed as limiting unless otherwise noted. Wherein either term is used, unless otherwise noted, the terms are used interchangeably and are intended to include the glycosylated as well as aglyconic materials.

Anthocyanidines, the aglyconic component of anthocyanins, have a basic structure as shown in Formula II

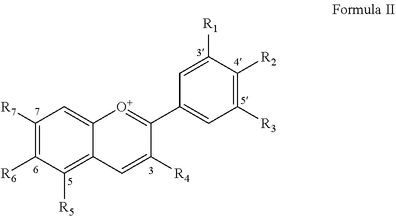

| | Anthocyanidin | | | | | | |
|---|---|---|---|---|---|---|---|
| | R₁ | R₂ | R₃ | R₄ | R₅ | R₆ | R₇ |
| Auantinidin | H | OH | H | OH | OH | OH | OH |
| Cyanidin | OH | OH | H | OH | OH | H | OH |
| Delphinidin | OH | OH | OH | OH | OH | H | OH |
| Europinidin | OCH₃ | OH | OH | OH | OCH₃ | H | OH |
| Luteolinidin | OH | OH | H | H | OH | H | OH |
| Pelargonidin | H | OH | H | OH | OH | H | OH |
| Malvidin | OCH₃ | OH | OCH₃ | OH | OH | H | OH |
| Peonidin | OCH₃ | OH | H | OH | OH | H | OH |
| Petunidin | OH | OH | OCH₃ | OH | OH | H | OH |
| Rosinidin | OCH₃ | OH | H | OH | OH | H | OCH₃ |

where R₁ through R₇ provide representative examples of anthocyanidins

The glycosylated forms of anthocyanins are more water soluble and stable than anthocyanidins. Anthocyanosides are classified by the number of glycosyl units they contain. Monoglycosides include one saccharidic moiety, which is primarily attached to the 3-hydroxyl group of the aglycon. Diglycosides generally contain two monosaccharides at the 3 and 5 hydroxy positions and occasionally at the 3 and 7 hydroxyl positions. Triglycosides have attachment generally where there are two units at the 3 position and one at the C-5 or C-7 position. Glycosylations at the 3′, 4′ and/or 5′ positions are also possible.

The most common sugars of anthocyanins include the monosaccharides glucose, rhamnose, galactose, arabinose and xylose. The di- and trisaccharides found most often in anthocyanins are rutinose, sophorose, sambubiose and glucorutinose.

Anthocyanins can be acylated through one or more hydroxyls with a carboxylic acid. The acids are most commonly linked to the 6 position of the monosaccharide but the 2, 3 and 4 positions of the monosaccharides are also possible. Common aliphatic acids include malonic, acetic, malic, succinic, and oxalic acids. Common aromatic phenolic acids and aliphatic dicarboxylic acids include coumaric, caffeic, sinapic, ferulic, oxalic, malonic, malic, succinic and gallic acid.

The term “extract” is intended to mean anthocyanin materials obtained from plant sources, such as leaves, twigs, bark, roots, stem, seeds, flowers, berries, fruit, for example, by routine isolation methods from suitable plants sources noted, but not limited to, those described herein. There are various methods for the extraction of anthocyanins known to those of skill in the art. Some of these methods are described in, for example, U.S. Pat. No. 5,817,354; U.S. Pat. No. 5,200,186; U.S. Pat. No. 5,912,363; U.S. Pat. No. 4,211,577; U.S. Pat. No. 4,302,200 (each incorporated herein by reference).

Examples of suitable anthocyanin-containing plants include, but are not limited to, fruits, vegetables, flowers and other plants selected from the group consisting of Acer macrophyllum, Acer platanoides, acerola, Ajuga reptans, apple, apricot, Artict bramble, avocado, banana, barberry, barley, Begonia semperfiorens, Bellis perennis, Bletilla striata, bilberry, black beans, black soybeans, black, blue and purple potatoes, blackberry, blueberry, bog whortleberry, boysenberry, buckwheat, cacao, Camellia sinensis, canarygrass, Caucasian blueberry, Chimonanthus praecox, celery, Cerasus avium, cherry, cherry laurel, chicory, chive, chokeberry, Cornelian cherry, cornflower, cotoneaster, cowberry, cranberry, crowberry, chrysanthemum, Cynomorium coccineum, Dahlia variabilis, danewort, deerberry, Dendrobium, dwarf dogwood, Echinacea purpea, eggplant, elderberry, fababean, Fatsia japonica, feijoa, fig, garlic, gerbera, ginseng, Globe artichoke, gooseberry, grapes, guava, hawthorn, hibiscus or roselle, Hibiscus Sabdaiffa, highbush blueberry, hollyhock, honeysuckle, Ipomoea purpurea, Iris ensata, Java plum, Jerusalem artichoke, kokum, Laeliocattleya, lentil, loganberry, lupine, lychee, maize, mango, mangosteen, maqui, Matthiola incana, meconopsis, Metrosideros excelsa, millet, mountain ash berry, mulberry, myrtle berry, olive, onion, orange, ornamental cherry, passion fruit, pea, peach, peanut, pear, perilla, petunia, Phalaenopsis, Phalsa, Pharbitis, Pineapple, pistachio, plum, pomegranate, Phragmites australis, purple carrot, quince, rabbiteye blueberry, radish, red and black currant, red and black raspberry, red cabbage, rice, rhubarb, rosehip, rye, saffron, sarracenia, sheepberry, Sophronitis coccinea, sorghum, sparkleberry, strawberry, Fragada Vesca, sugarcane, sunflower, sweet cherry, sweet potato, tamarillo, tamarind, taro, tart cherry, Tulip greigii, turnip, water lily, Weigela, wheat, wild rice, Verbena hybrida, yam and mixtures thereof.

Although there are literally thousands of anthocyanin extracts, all of which should be considered included within the realm of this specification, suitable examples of anthocyanin extracts of particular interest include bilberry extract, blackcurrant extract, cranberry extract, black soybean extract, cowberry extract, blueberry extract and mixtures of two or more thereof.

Typically the extract is concentrated by various methods to provide a solution enriched in anthocyanins. For example, ultrafiltration can be used to remove unwanted components by molecular weight cut offs. The retentate from the filtration can be stored as a liquid or, for example, can then be further concentrated into a powder by spray drying, freeze drying, flash drying, fluidized bed drying, ring drying, tray drying, vacuum drying, radio frequency drying or microwave drying. Ultimately, the extract should contain at least 10% by weight anthocyanin content. Commercially available anthocyanins can be obtained from sources such as Artemis International, Fort Wayne, Ind. Commercially obtained anthocyanin extracts should contain at least 10% by weight anthocyanin content. The extracts, therefore, contain anthocyanin(s) and other plant materials such as other flavinoids, sugars, etc.

Anthocyanin extracts can be further purified by one or more methods known in the art, such as chromatography, gel chromatography, high performance liquid chromatography, crystallization, affinity chromatography, partition chromatography and the like. Identification of the particular anthocyanin(s) can be accomplished by methods know to those skilled in the art and include ¹H NMR, chemical degradation, chromatography and spectroscopy, especially homo- and heteronuclear two-dimensional NMR techniques for the characterization of the isolated anthocyanin compounds.

The term “purified” or “isolated” is used in reference to the purification and/or isolation of one or more anthocyanins from an anthocyanin extract as described above. Again using conventional methods known in the art, various components of the anthocyanin extract can be separated into purified materials. In one aspect of the invention, the anthocyanin(s) of the extract are substantially purified and isolated by techniques known in the art. The purity of the purified compounds is generally at least about 90%, preferably at least about 95%, and most preferably at least about 99% and even more preferably at least about 99.9% (e.g. about 100%) by weight.

In accordance with the present invention, the anthocyanin extract contains one or more anthocyanins and/or anthocyanidins selected from the group consisting of peonidin, cyanidin, pelargonidin, delphinldin, petunidin, malvidin, apigenindin, auratinidin, capensinidin, europinidin, hirsutidin, 6-hydroxycyanidin, luteolinidin, 5-methylcyanidin, pulchellidin, rosinidin, tricetnidin, derivatives and mixtures thereof. In one embodiment, the anthocyanins and anthocyanidins are selected from the group consisting of cyanidin, peonidin, malvidin, petunidin, delphinidin, their glycoside derivatives, and mixtures thereof. In yet another embodiment, the extract contains at least one cyanidin-based anthocyanin.

The term “anthocyanin” as used herein is intended to refer not only to monomeric anthocyanins, but also refers to dimeric and polymeric (i.e. containing from 3 to 20 anthocyanidin monomer residues) forms of anthocyanins and to leucoanthocyanidins (also known as flavan-3,4-diols). The anthocyanins can comprise substitutions (e.g. alkyl, alkoxy groups etc.) and in particular can be O-glycosylated, as described above.

The anthocyanin in the composition can be a single anthocyanin, or comprise a mixture of anthocyanins. In particular, the anthocyanin is selected from the group consisting of: malvidin, cyanidin, delphinidin, paeonidin, pelargonidin and petunidin, and glycosides thereof A typical example is malvin (malvidin diglucoside) chloride, which is commercially available in a purified form. Alternatively the anthocyanin can be obtained by extracting anthocyanin containing plants such as grape, black carrot, red cabbage, blackberry, blackcurrent, cranberry and the like as described above.

In one aspect, the ratio of stabilizing compound to anthocyanin extract is about 0.1 to about 10, more particularly about 0.5 to about 5, and more particularly about 1 to about 1.

In still another aspect, the stabilized anthocyanin extract is an anthocyanoside.

In still yet another aspect, the stabilized anthocyanin extract is an anthocyanidin.

In still other aspects of the invention, the stabilized anthocyanin extract includes one or more anthocyanosides that are glycosidse of perlargonidin, peonidin, cyanidin, malvidin, petunidin, delphinidin.

In one embodiment, a melanin can be used as the antioxidant. Melanin is a class of compounds found in plants, animals, and protists, where it serves predominantly as a pigment. The class of pigments are derivatives of the amino acid tyrosine. The most common form of biological melanin is eumelanin, a brown-black polymer of dihydroxyindole, dihydroxyindole carboxylic acid, and their reduced forms. Another common form of melanin is pheomelanin, a red-brown polymer of benzothiazine units. Other common melanins include sepiamelanins, neuromelanins and allomelanins.

In another aspect, a sesame extract (a melanin) can be used as the antioxidant and is characterized by having a UV-VIS spectrum with an absorbance between about 0.1 to about 0.16 at 500 nm at a concentration at less than 0.04 mg/ml in an aqueous solution.

The sesame extract has a molecular weight of between about 264 and about 5400 daltons, more particularly between about 264 and about 2000 daltons, even more particularly between about 400 and about 1600 daltons, more particularly between about 264 and about 1600 daltons, more particularly between about 532 and 1064 daltons, and all ranges there between from about 264 to about 5400 daltons.

The sesame extract, a reduced melanin pigment can be characterized by the elemental composition of the reduced melanin. The carbon content of the reduced melanin is between about 35 percent and about 57 percent by weight, the hydrogen content of the reduced melanin is between about 5 percent and about 10 percent by weight and the nitrogen content is between about 5 and about 10 percent by weight. The oxygen content, if present, of the reduced melanin is between about 33 percent and about 37 percent by weight.

In one aspect, the nitrogen content (if present) of the reduced melanin is between about 5 percent and about 10 percent by weight.

In another aspect, the sulfur content (if present) of the reduced melanin is less than about 1 percent by weight.

The black sesame seed useful in the invention is the seed from the plant Sesamum indicum. Generally, the reduced melanin pigment is derived from a seed shell or hull particularly from a sesame seed that includes melanin. Alternatively, twigs, bark, branches, leaves and/or roots to the sesame plant can be used to obtain the starting material melanin. The seed shell or hull of the black sesame seed is most abundant with the desired melanin used to provide the reduced melanin.

In one aspect the reduced melanin has an infrared spectra that exhibits distinctive absorption bands at 3400 cm⁻¹ (phenolic —OH stretches), at about 2950 cm⁻¹ (aliphatic stretches), at about 1600 to about 1650 cm⁻¹ (aromatic C═C stretches), at about 1380 to about 1400 cm⁻¹ (phenolic C—OH, indolic and phenolic NH stretches), at about 1260 cm⁻¹ (phenolic COH) and at about 1170 cm⁻¹ (C═O stretch).

The sesame extract can be prepared in the following manner. The methods include mixing an aqueous base and pulverized sesame seed at an elevated temperature followed by cooling of the resultant mixture to room temperature. The resultant mixture is filtered and rinsed with water to provide a filtrate. The filtrate is treated with an aqueous acid and a reducing agent is added to the filtrate until evolution of gas ceases and the sesame extract is formed.

The concentration of the base in the aqueous base is between about 0.5 percent and about 2 percent by weight. Suitable bases include amines and various metal hydroxides, such as potassium hydroxide and sodium hydroxide. Generally the ratio of aqueous base to sesame seed is about 5:1 on a weight basis and generally the elevated temperature of the first step is between about 40° C. and about 80° C., e.g., 60° C. The mixture is generally heated for between about 0.5 hours and about 5 hours, e.g., 3 hours.

The treated filtrate of the process is agitated (e.g., stirred or sonicated) prior to addition of the reducing agent. Suitable reducing agents include dihydrolipoic acid, sodium borohydride, or other reducing agents having an —SH group. The reducing agent is added to the filtrate at temperature between about 10° C. and about 35° C., more particularly at about 25° C.

The present invention also provides an alternative method to prepare a reduced melanin pigment (sesame extract). The method includes mixing an aqueous base and pulverized sesame seed at or below room temperature. The mixture is filtered and rinsed with water to provide a filtrate. The filtrate is treated with an aqueous acid at or below room temperature to a pH of between about below 7 and a pH of about 1. The resultant reduced melanin pigment collected.

The concentration of the base in the aqueous base in step is between about 0.5 percent and about 2 percent by weight (pH above about 10, for example between about 10 and about 14, and for example, a pH of about 12). Suitable bases include amines and various metal hydroxides, such as potassium hydroxide and sodium hydroxide. Generally the ratio of aqueous base to sesame seed is about 5:1 on a weight basis and generally the temperature of the mixture is maintained below about 35° C., e.g., 25° C. The mixture of step a) is mixed for between about 0.5 hours and about 48 hours, for example, about 24 hours.

The aqueous acid can be any organic or mineral acid, such as acetic acid, hydrochloric acid, sulfuric acid, nitric acid, or phosphoric acid is about 15% acid by volume. The temperature of the filtrate is maintained below about 35° C., for example, 25° C. during and after the addition of the acid solution. Generally the pH is lowered below a pH of 7 and in particular to a pH of about 1.

The resultant reduced melanin pigment can be collected by various methods such as filtrations or by centrifugation.

Suitable procedures for the preparation of the reduced melanin can be found in U.S. patent application Ser. No. 12/510,395, filed Jul. 28, 2009 and U.S. Provisional Patent Application No. 61/083,994, filed Jul. 28, 2008, the contents of which are incorporated herein by reference.

In still another aspect, suitable examples of stabilizing compounds (also referred to as antioxidants) include yeast extract (e.g., beer yeast), dihydrolipoic acid, salts of dihydrolipoic acid such as amino acid salts, cysteine, derivatives of cysteine, such as N-acetylcysteine, glutathione, salts of glutathione, SH-proteinase such as papain, bromelain, ficin, ehymopapain and mixtures thereof, SH-metalloproteinases, peptides containing cysteine, peptides containing glutathioine, fermented oyster extract, fermented bean curd, thiolated chitosans, thiolated gelatins or mixtures thereof.

One example of an —SH bearing compound is dihydrolipoic acid. Dihydrolipoic acid (DHLA) is a constituent of cellular metabolism. DHLA has two thiol residues that make is susceptible to radical species, thus provides antioxidant functionality to the biomolecule. Oxidation reduction (redox reactions) involves the transfer of an electron from a donor to an acceptor. When the donor loses an electron, it is transformed from its reduced form to its oxidized form. When an acceptor gains an electron, it changes from its oxidized form to its reduced form.

Dihydrolipoic acid is the reduced (has electrons added) form of lipoic acid (thioctic acid). When DHLA is oxidized (has electrons removed) lipoic acid is produced. It should be understood that DHLA can be either the R or S enantiomer or it can be racemic. Likewise, lipoic acid can also be enantiomerically pure or racemic.

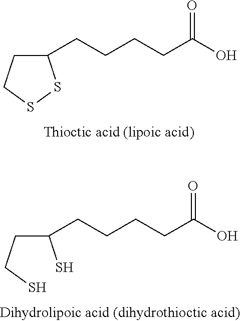

The term “coenzyme Q” or “ubiquinone” (CoQ-10) is used throughout the present specification to describe a group of lipid soluble benzoquinones involved in electron transport in mitochondrial preparations, i.e., in the oxidation of succinate or reduced nicotine adenine dinucleotide (NADH) via the cytochrome system. The compounds can be described as: coenzyme Qₙ where n is 1-12 or ubiquinone (x) in which x designates the total number of carbon atoms in the side chain and can be any multiple of 5. Differences in properties are due to the difference in the chain length. In particular, ubiquinone for use in the present invention is the reduced form of coenzyme Q10, known as ubiquinol. Therefore, the term CoQ-10 includes all variations where n is from 1 to 12. Likewise, reduced CoQ-10 also includes all variation where n is from 1 to 12.

The term “ubiquinol” is used throughout the specification to describe the reduced form of coenzyme Qₙ that is used as the active agent in compositions according to the present invention. In ubiquinol, the quinone ring of coenzyme Qₙ is reduced such that the structure of the compound appears as set forth below. In one aspect, ubiquinol, n is preferably 10 and is derived from coenzyme Q₁₀. It should be understood, that throughout this specification, reference to CoQ-10 and reduced CoQ-10 refers to all possible derivatives where n is as detailed above.

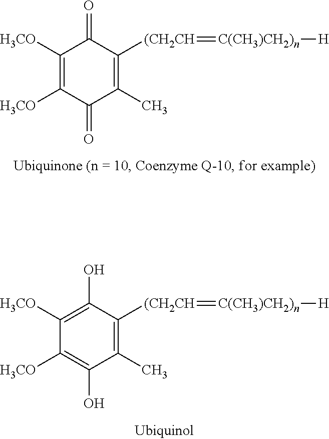

While not being limited by way of theory, it is believed that effective concentrations of the antioxidants described herein e.g., anthocyanins, a melanin or sesame seed extract, a compound having at least one thiol (—SH) group or mixtures thereof, convert substantially all ubiquinone to ubiquinol. In other embodiments, effective concentrations of DHLA also help prevent ubiquinol from being oxidized to ubiquinone, or alternatively reduce any ubiquinone, which has been oxidized from ubiquinol during storage of the compositions according to the present invention.

The formulations of the invention are considered dietary supplements useful to the increase the amounts of reduced CoQ-10 and/or additional antioxidants in individuals in need thereof.

The formulations of the invention can also be used in cosmetic products.

Alternatively, the formulations of the invention are also considered to be nutraceuticals. The term “nutraceutical” is recognized in the art and is intended to describe specific chemical compounds found in foods that may prevent disease. Reduced CoQ-10 and antioxidants are such compounds.

The formulations of the invention can further include various ingredients to help stabilize, or help promote the bioavailability of the CoQ-10, or serve as additional nutrients to an individual's diet. Suitable additives can include vitamins and biologically-acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals may be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Vitamin(s), if present, are present in the composition of the invention in an amount ranging from about 5 mg to about 500 mg. More particularly, the vitamin(s) is present in an amount ranging from about 10 mg to about 400 mg. Even more specifically, the vitamin(s) is present from about 250 mg to about 400 mg. Most specifically, the vitamin(s) is present in an amount ranging from about 10 mg to about 50 mg. For example, B vitamins are in usually incorporated in the range of about 1 milligram to about 10 milligrams, i.e., from about 3 micrograms to about 50 micrograms of B12. Folic acid, for example, is generally incorporated in a range of about 50 to about 400 micrograms, biotin is generally incorporated in a range of about 25 to about 700 micrograms and cyanocobalamin is incorporated in a range of about 3 micrograms to about 50 micrograms.

Mineral(s), if present, are present in the composition of the invention in an amount ranging from about 25 mg to about 1000 mg. More particularly, the mineral(s) are present in the composition ranging from about 25 mg to about 500 mg. Even more particularly, the mineral(s) are present in the composition in an amount ranging from about 100 mg to about 600 mg.

Various additives can be incorporated into the present compositions. Optional additives of the present composition include, without limitation, phospholipids, L-carnitine, starches, sugars, fats, antioxidants, amino acids, proteins, flavorings, coloring agents, hydrolyzed starch(es) and derivatives thereof or combinations thereof.

As used herein, the term “phospholipid” is recognized in the art, and refers to phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins.

As used herein, the term “antioxidant” is recognized in the art and refers to synthetic or natural substances that prevent or delay the oxidative deterioration of a compound. Exemplary antioxidants, further encompassing the anthocyanins, —SH containing molecules and/or sesame extracts described herein, include tocopherols, flavonoids, catechins, superoxide dismutase, lecithin, gamma oryzanol; vitamins, such as vitamins A, C (ascorbic acid) and E and beta-carotene; natural components such as camosol, carnosic acid and rosmanol found in rosemary and hawthorn extract, proanthocyanidins such as those found in grapeseed or pine bark extract, and green tea extract.

The term “flavonoid” as used herein is recognized in the art and is intended to include those plant pigments found in many foods that are thought to help protect the body from cancer. These include, for example, epi-gallo catechin gallate (EGCG), epi-gallo catechin (EGC) and epi-catechin (EC).

Any dosage form, and combinations thereof, are contemplated by the present invention. Examples of such dosage forms include, without limitation, chewable tablets, elixirs, liquids, solutions, suspensions, emulsions, capsules, soft gelatin capsules, hard gelatin capsules, caplets, lozenges, chewable lozenges, suppositories, creams, topicals, ingestibles, injectables, infusions, health bars, confections, animal feeds, cereals, cereal coatings, and combinations thereof. The preparation of the above dosage forms are well known to persons of ordinary skill in the art.

For example, health bars can be prepared, without limitation, by mixing the formulation plus excipients (e.g., binders, fillers, flavors, colors, etc.) to a plastic mass consistency. The mass is then either extended or molded to form “candy bar” shapes that are then dried or allowed to solidify to form the final product.

CoQ-10 has been implicated in various biochemical pathways and is suitable for the treatment of cardiovascular conditions, such as those associated with, for example, statin drugs that effect the body's ability to product CoQ-10 naturally. CoQ-10 has also been implicated in various periodontal diseases. Furthermore, CoQ-10 has been implicated in mitochondrial related diseases and disorders, such as the inability to product acetyl coenzyme A, neurological disorders, for example, such as Parkinson's disease and, Prater-Willey syndrome, migraine headaches and headaches. Therefore, the present invention provides compositions that can be used to treat one or more of such disease states or conditions.

The following paragraphs enumerated consequently from 1 through 34 provide for various aspects of the present invention. In one embodiment, in a first paragraph (1), the present invention provides a reduced coenzyme Q-10 (CoQ-10) composition, comprising reduced coenzyme Q10 and an anthocyanin extract.

2. A composition comprising reduced coenzyme Q10, oxidized coenzyme Q10 and an anthocyanin extract.

3. The composition according to paragraph 1 or 2, further comprising a compound having at least one thiol (—SH) group.

4. The composition according to paragraph 3, wherein the compound having at least one —SH group is a yeast extract, DHLA, a derivative of DHLA, cysteine, a derivative of cysteine, glutathione, a derivative of glutathione, a thiol group containing protein, or mixtures thereof.

5. The composition according to any of paragraphs 1 through 4, wherein the anthocyanin extract is bilberry extract, blackcurrant extract, cranberry extract, black soybean extract, cowberry extract, blueberry extract or mixtures of two or more thereof.

6. A method to stabilize reduced coenzyme Q10 comprising the step of combining an anthocyanin extract and reduced coenzyme Q10.

7. A method to stabilize reduced coenzyme Q10 comprising the step of combining an anthocyanin extract, a reduced coenzyme Q10 and a compound having at least one thiol (—SH) group.

8. The method according to paragraph 7, wherein the compound having at least one —SH group is a yeast extract, DHLA, a derivative of DHLA, cysteine, a derivative of cysteine, glutathione, a derivative of glutathione, a thiol group containing protein, or mixtures thereof.

9. The method according to any of paragraphs 6 through 8, wherein the anthocyanin extract is bilberry extract, blackcurrant extract, cranberry extract, black soybean extract, cowberry extract, blueberry extract or mixtures of two or more thereof. 10. A composition comprising reduced coenzyme Q10 and a melanin, with the proviso the melanin is not a reduced sesame seed extract. 11. A composition comprising reduced coenzyme Q10, oxidized coenzyme Q10 and a melanin, with the proviso the melanin is not a reduced sesame seed extract.

12. The composition according to either paragraph 10 or 11, further comprising a compound having at least one thiol (—SH) group.

13. The composition according to paragraph 12, wherein the compound having at least one —SH group is a yeast extract, DHLA, a derivative of DHLA, cysteine, a derivative of cysteine, glutathione, a derivative of glutathione, a thiol group containing protein, or mixtures thereof.

14. The composition according to any of paragraphs 10 through 13, wherein the melanin is derived from sesame seed hull.

15. A method to stabilize reduced coenzyme Q10 comprising the step of combining melanin and reduced coenzyme Q10.

16. A method to stabilize reduced coenzyme Q10 comprising the step of combining melanin, reduced coenzyme Q10 and a compound having at least one thiol (−5H) group.

17. The method according to paragraph 16, wherein the compound having at least one —SH group is a yeast extract, DHLA, a derivative of DHLA, cysteine, a derivative of cysteine, glutathione, a derivative of glutathione, a thiol group containing protein, or mixtures thereof.

18. The method according to any of paragraphs 15 through 17, wherein the melanin is derived from sesame seed hull.

19. A composition comprising reduced coenzyme Q10 and a compound having at least one thiol (—SH) group, with the proviso that the —SH containing group is not DHLA.

20. A composition comprising reduced coenzyme Q10, oxidized coenzyme Q10 and a compound having at least one thiol (—SH) group, with the proviso that the —SH containing group is not DHLA.

21. The composition according to either paragraph 19 or 20, wherein the compound having at least one thiol (—SH) group is a yeast extract, cysteine, a derivative of cysteine, glutathione, a derivative of glutathione, a thiol group containing protein, or mixtures thereof.

22. The composition according to paragraph 19 through 21, further comprising melanin or an anthocyanin extract.

23. The composition according to paragraph 22, wherein the anthocyanin extract is bilberry extract, blackcurrant extract, cranberry extract, black soybean extract, cowberry extract, blueberry extract or mixtures of two or more thereof.

24. The composition according to paragraph 22, wherein the melanin is derived from sesame seed hull.

25. A method to stabilize reduced coenzyme Q10 comprising the step of combining a compound having at least one thiol (—SH) group and reduced coenzyme Q10.

26. A method to stabilize reduced coenzyme Q10 comprising the step of combining melanin, reduced coenzyme Q10 and a compound having at least one thiol (—SH) group.

27. A method to stabilize reduced coenzyme Q10 comprising the step of combining anthocyanin extract, reduced coenzyme Q10 and a compound having at least one thiol (—SH) group.

28. The method according to any of paragraphs 25 through 27, wherein the compound having at least one thiol (—SH) group is a yeast extract, DHLA, a derivative of DHLA, cysteine, a derivative of cysteine, glutathione, a derivative of glutathione, a thiol group containing protein, or mixtures thereof.

29. The method according to either paragraphs 26 or 28, wherein the melanin is derived from sesame seed hull.

30. The method according to either paragraph 27 or 28, wherein the anthocyanin extract is bilberry extract, blackcurrant extract, cranberry extract, black soybean extract, cowberry extract, blueberry extract or mixtures of two or more thereof.

31. The composition of any of paragraphs 1, 10 or 19, wherein the percentage of reduced coenzyme Q10 is at least 95% by weight.

32. The composition of any of paragraphs 1, 10 or 19, wherein the amount of non-reduced coenzyme Q10 is not detectable by HPLC.

33. The composition of any of paragraphs 2, 11 or 20, wherein the percentage of reduced coenzyme Q10 is between about 20% and 99.99% by weight and the percentage of non-reduced coenzyme Q10 is between about 80% and about 0.01% by weight. For example, exemplary ranges include from about 20 to 99.99% (by weight) reduced coenzyme Q10 with the remainder being coenzyme Q10, about 50%, about 60%, about 80%, about 90% and about 95% reduced coenzyme Q10 with the remainder being coenzyme Q10 by weight based on 100%.

34. The method of any of paragraphs 6 through 9, 15 through 18 or 25 through 30, wherein the percentage of reduced coenzyme Q10 is between about 20% and 99.99% by weight and the percentage of non-reduced coenzyme Q10 is between about 80% and about 0.01% by weight.

The following examples are intended to be illustrative only and should not be considered limiting.

### Example 1

### Preparation of CoQH10 with 90% of reduced form

Incubation: 41.3L absolute ethanol was added to a glass reactor, under stirring, 5.50 kg oxidized form CoQ10 (6.25 mol) and 3.44 kg DHLA (16.5 mol) were mixed in the reactor. When the reaction mixture was heated to 45° C. and the CoQ10 was completely dissolved, the reaction system was maintained for 20 minutes, and then 0.344 L solution of sesame pigment (sesame pigment prepared according to U.S. patent application Ser. No. 12/510,395; 0.05 g/ml NaOH in water, containing 0.05 g/ml sesame pigment) was slowly added. After addition of pigment solution was finished, the reactor was sealed and was maintained without temperature change for 1 hour. The temperature then decreased to 40° C. within 1 hour and was held at 40° C. for 20 hours.

Cooling process: When the incubation at 40° C. was finished, 2.06 kg pure water was added and the mixture was stirred for 1 hour. The reaction mixture temperature decreased to 20° C. within 3 hours and was maintained at 20° C. for 2 hours. The temperature of the reaction mixture was then decreased to 15° C. within 1 hour and maintained for 1 hour.

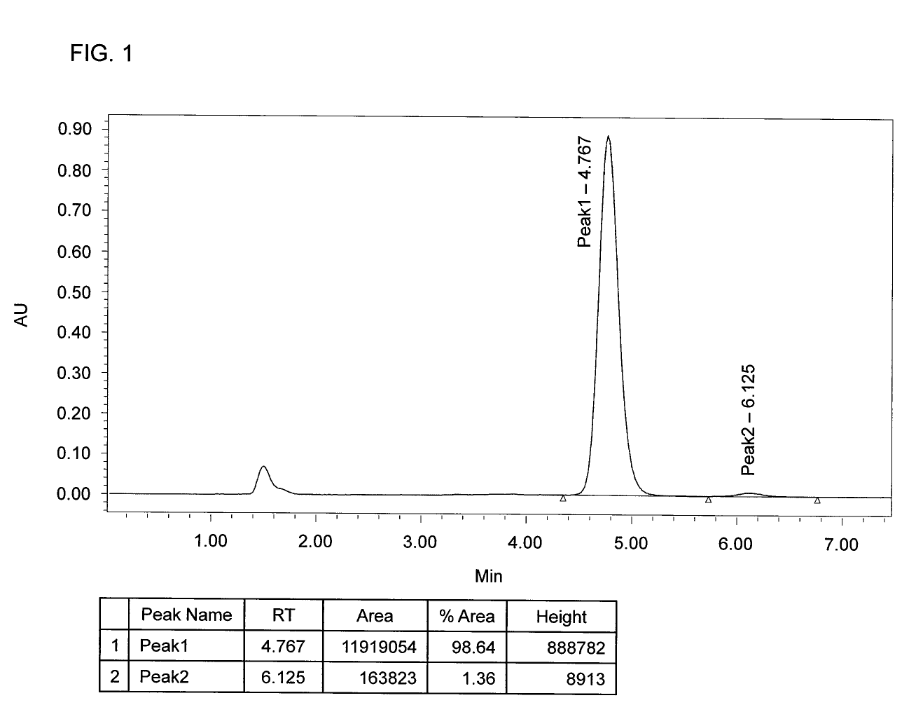

After the cooling process, the reduced CoQ10 was separated from the solution. After rapid filtration within 1 hour, the filter cake was washed with 4.13 L cooled 95% ethanol. The filter cake was dried under a nitrogen atmosphere for 4 hours and was then vacuum dried at about 25° C. and 1000 Pa to completely remove the ethanol. The yield of reduced CoQ10 was 5.3 kg with a purity of 98.64% ubiquinol determined by HPLC as noted in FIG. 1. The HPLC conditions were:

Instrumentation: Waters 600 pump with 717 autosampler and 2996 DAD.

Mobile Phase: ethanol:methanol=25:75

UV-detection: at 210 nm

Column: Symmetry RP-C18 (waters) 3.9 mm×150 mm, 5 μm

Flow rate: 1.0 ml/min

Temperature: 25° C.

Injection volume: 0.5 μl

### Example 2

### Preparation of QH10 with 50% of reduced form

The QH10 of example 1 was taken as starting material, and then was recrystallized with oxidized form CoQ10 by ethanol-water system. QH10 with 50% of reduced form was prepared.

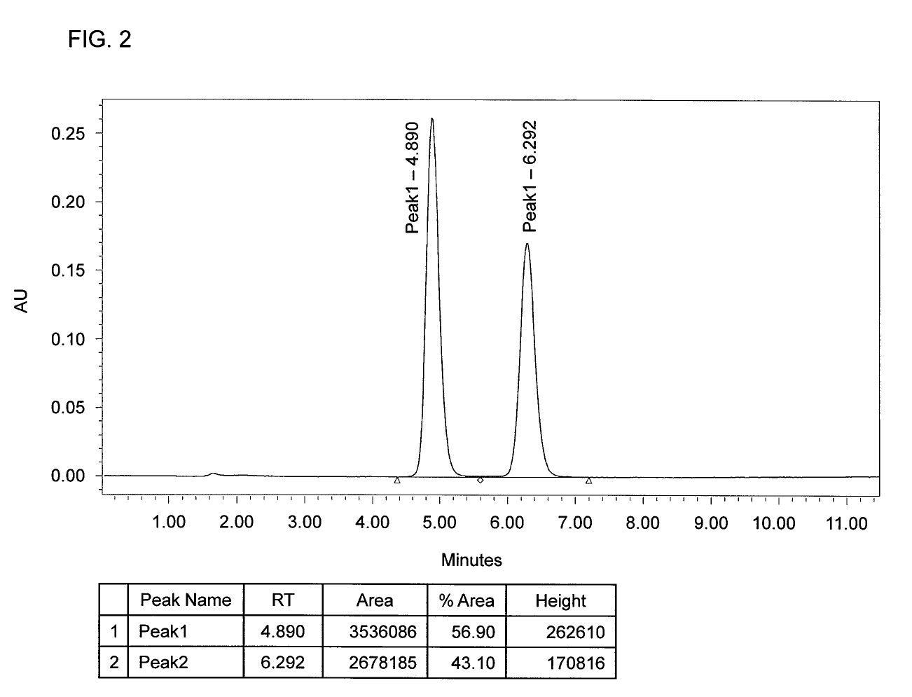

In a glass reactor, 41.3 L absolute ethanol was added, then 3.06 kg reduced form CoQ10 (from example 1) and 2.24 kg oxidized form CoQ10 were mixed in the reactor. The mixture was heated to 45° C. and the CoQ10 was completely dissolved. The reaction mixture was balanced for 20 minutes, and then 2.06 kg pure water was slowly added under stirring within 1 hour. The reaction mixture decrease to 20° C. within 3 hours and was maintained at 20° C. for 2 hours, then decreased to 15° C. within 1 hour and maintained at 15° C. for 1 hour. The CoQ10 crystals were separated from the solution. After filtration within 1 hour, the filter cake was washed with 4.13 L cooled 95% ethanol. The filter cake was dried under a nitrogen atmosphere for 4 hours and was then vacuum dried at about 25° C. and 1000 Pa to completely remove ethanol. 5.1 kg ubiquinol 56.9%/CoQ10 43.1% determined by HPLC was afforded as noted in FIG. 2. The HPLC conditions were the same as provided in Example 1.

Stability Test 1

The following example provides good stability of QH10 powder of Examples 1 and 2, when compared with a commercial reduced CoQ10.

Commercial reduced CoQ10 powder is not stable in the ambient air. Generally, exposure to air can cause the material to become oxidized, resulting in below 20% of reduced form of CoQ10 on a weight basis. Surprisingly, the present invention provides the QH10 powder is fairly stable in the ambient air.

As noted below, in Example 1, the QH10 powder contained a minor amount of DHLA and a trace amount (below 0.25% by weight) of sesame pigment (melanin). The tests below compared QH10 powder of the invention with QH10 powder without DHLA (by removing DHLA).

Sample preparation:

(1) QH10 powder of example 1, with a content of 0.9% of DHLA;

(2) Non-DHLA fully reduced powder. QH10 powder of example 1 as starting material, DHLA was removed by recrystallization with ethanol. DHLA is less than 0.1% by weight.

(3) Non-DHLA fully reduced powder. QH10 powder of example 2 as starting material, DHLA was removed by recrystallization with ethanol. DHLA is less than 0.1% by weight.

Treatment:

The three samples were kept in containers with exposure to ambient air for 15 days, then the residual ratio of reduced form of CoQ 10 was determined by HPLC analysis. HPLC conditions were the same as in Example 1.

Results:

| Sample | Percentage of reduced | Percentage of reduced | Percentage of |
|---|---|---|---|
| No. | form CoQ10 at 0 day | form CoQ10 at 15 day | degradation |
| (1) | 98 | 97 | 1 |
| (2) | 96 | 89 | 7 |
| (3) | 57 | 48 | 9 |

Stability Test 2

This example showed good reducing ability of anthocyanin to Q10.

Treatment:

100 mg oxidized form coenzyme Q10 was mixed with 500 mg/1000 mg bilberry extract (Omya-Peralta GmbH; Lot No.: BB0823-1, containing about 36% anthocyanins), and then 50 ml absolute ethanol was added. The reaction mixture was treated by ultrasonication for about 30 minutes and then the reactor was sealed and stored for 5 days at ambient conditions.

Reduced coenzyme Q10 was determined by HPLC analysis as noted in Example 1.

Results:

### (1) Reduction Ratios and Anthocyanins Dosage

| Dosage of bilberry extract/mg | reduced ratio of Q10/% |
|---|---|
| 500 | 70.2 |
| 1000 | 70.3 |

(2) Reduction Ratios and Reaction Time with 1000 Mg Bilberry Extract

| Reaction time/day(s) | Reduced ratio of Q10/% |
|---|---|
| 1 | 20.8 |
| 5 | 70.3 |

Stability Test 3

This example showed good stability of QH-10 (reduced coQ-10) with anthocyanin.

Treatment:

50 mg reduced form of coenzyme Q10 (97% of purity of reduced form of Q10) was mixed with 50 mg protective agent, and then 50 ml absolute ethanol was added. The reaction mixture was treated by ultrasonication for about 30 minutes, and then the reactor was sealed and stored for 16 hours at ambient conditions. Reduced coenzyme Q10 was determined by HPLC analysis as noted in Example 1.

Protective agent:

bilberry extract (Omya-Peralta GmbH; Lot No.: BB0823-1, containing about 36% anthocyanins)

blackcurrant extract (Omya-Peralta GmbH; Lot No.; BC6006, containing about 36% anthocyanins)

Results:

| Protective agent | Residual percentage of Reduced Q10% |
|---|---|
| No protective agent | 0.5 |
| Bilberry extract | 96.5 |
| Black currant extract | 95.9 |

Example of Reduced Sesame (Melanin)

5 g sodium hydroxide were dissolved in 500 ml de-ionized water, and then added to 100 g pulverized black sesame seed. The mixture was stirred and heated to 60° C. The reaction mixture was maintained at 60° for 3 hours and then cooled to room temperature. The black mixture was filtered and the filter cake was washed with 100 ml de-ionized water. The filtrate and rinse solution was treated with an aqueous acid solution (pH about 1). The solution was sonicated for 10 minutes at a temperature of 25° C. Potassium borohydride was added slowly while maintaining the reaction temperature below or at 25° C. The mixture was allowed to stand for about 25 minutes until gas evolution ceased. The potassium borohydride can be replaced by other reducing agents, such as DHLA. Potassium borohydride can be removed by water washes. DHLA can be removed by washing the product with ethanol.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A composition comprising reduced coenzyme Q10 and an anthocyanin extract.

2. A composition comprising reduced coenzyme Q10, oxidized coenzyme Q10 and an anthocyanin extract.

3. The composition according to claim 1, further comprising a compound having at least one thiol (—SH) group.

4. The composition according to claim 3, wherein the compound having at least one —SH group is a yeast extract, DHLA, a derivative of DHLA, cysteine, a derivative of cysteine, glutathione, a derivative of glutathione, a thiol group containing protein, or mixtures thereof.

5. The composition according to claim 1, wherein the anthocyanin extract is bilberry extract, blackcurrant extract, cranberry extract, black soybean extract, cowberry extract, blueberry extract or mixtures of two or more thereof.

6. A method to stabilize reduced coenzyme Q10 comprising the step of combining an anthocyanin extract and reduced coenzyme Q10.

7. A method to stabilize reduced coenzyme Q10 comprising the step of combining an anthocyanin extract, a reduced coenzyme Q10 and a compound having at least one thiol (—SH) group.

8. The method according to claim 7, wherein the compound having at least one —SH group is a yeast extract, DHLA, a derivative of DHLA, cysteine, a derivative of cysteine, glutathione, a derivative of glutathione, a thiol group containing protein, or mixtures thereof.

9. The method according to claim 6, wherein the anthocyanin extract is bilberry extract, blackcurrant extract, cranberry extract, black soybean extract, cowberry extract, blueberry extract or mixtures of two or more thereof.

10. A composition comprising reduced coenzyme Q10 and a melanin, with the proviso the melanin is not a reduced sesame seed extract.

11. A composition comprising reduced coenzyme Q10, oxidized coenzyme Q10 and a melanin, with the proviso the melanin is not a reduced sesame seed extract.

12. The composition according to claim 10, further comprising a compound having at least one thiol (—SH) group.

13. The composition according to claim 12, wherein the compound having at least one —SH group is a yeast extract, DHLA, a derivative of DHLA, cysteine, a derivative of cysteine, glutathione, a derivative of glutathione, a thiol group containing protein, or mixtures thereof.

14. The composition according to claim 10, wherein the melanin is derived from sesame seed hull.

15. A method to stabilize reduced coenzyme Q10 comprising the step of combining melanin and reduced coenzyme Q10.

16. A method to stabilize reduced coenzyme Q10 comprising the step of combining melanin, reduced coenzyme Q10 and a compound having at least one thiol (—SH) group.

17. The method according to claim 16, wherein the compound having at least one —SH group is a yeast extract, DHLA, a derivative of DHLA, cysteine, a derivative of cysteine, glutathione, a derivative of glutathione, a thiol group containing protein, or mixtures thereof.

18. The method according to claim 15, wherein the melanin is derived from sesame seed hull.

19. A composition comprising reduced coenzyme Q10 and a compound having at least one thiol (—SH) group, with the proviso that the —SH containing group is not DHLA.

20. A composition comprising reduced coenzyme Q10, oxidized coenzyme Q10 and a compound having at least one thiol (—SH) group, with the proviso that the —SH containing group is not DHLA.

21. The composition according to claim 19, wherein the compound having at least one thiol (—SH) group is a yeast extract, cysteine, a derivative of cysteine, glutathione, a derivative of glutathione, a thiol group containing protein, or mixtures thereof.

22. The composition according to claim 19, further comprising melanin or an anthocyanin extract.

23. The composition according to claim 22, wherein the anthocyanin extract is bilberry extract, blackcurrant extract, cranberry extract, black soybean extract, cowberry extract, blueberry extract or mixtures of two or more thereof.

24. The composition according to claim 22, wherein the melanin is derived from sesame seed hull.

25. A method to stabilize reduced coenzyme Q10 comprising the step of combining a compound having at least one thiol (—SH) group and reduced coenzyme Q10.

26. A method to stabilize reduced coenzyme Q10 comprising the step of combining melanin, reduced coenzyme Q10 and a compound having at least one thiol (—SH) group.

27. A method to stabilize reduced coenzyme Q10 comprising the step of combining anthocyanin extract, reduced coenzyme Q10 and a compound having at least one thiol (—SH) group.

28. The method according to claim 25, wherein the compound having at least one thiol (—SH) group is a yeast extract, DHLA, a derivative of DHLA, cysteine, a derivative of cysteine, glutathione, a derivative of glutathione, a thiol group containing protein, or mixtures thereof.

29. The method according to claim 26, wherein the melanin is derived from sesame seed hull.

30. The method according to claim 27, wherein the anthocyanin extract is bilberry extract, blackcurrant extract, cranberry extract, black soybean extract, cowberry extract, blueberry extract or mixtures of two or more thereof.
